(19) United States
(12) Patent Application Publication
(10) Pub. No.: US 2008/0028637 A1
(43) Pub. Date: Feb. 7, 2008

(54) SHOE INSERT FOR COOLING FOOT

(76) Inventor: Eugene L. Benfatti, Roseland, NJ (US)

Correspondence Address: STANLEY H. KREMEN, 4 LENAPE LANE, EAST BRUNSWICK, NJ, 08816, US

(21) Appl. No.: 11/872,711

(22) Filed: Oct. 16, 2007

Related U.S. Application Data
(60) Provisional application No. 60/852,071, filed on Oct. 16, 2006.

(51) Int. Cl. A43B 13/20; A43B 13/38; A61F 5/14

(52) U.S. Cl. 36/43; 36/29; 36/153

(57) ABSTRACT

A shoe insert to be used for cooling the foot to be manufactured and sold as pairs of left and right shoe inserts. The insert has a number of components forming a sandwich. The insert has the shape of a shoe, and is partly adjustable in size to fit a range of shoe sizes. So, the inserts may be sold in sizes, for example, small, medium, and large, and these sizes may then be fine tuned to fit a sub-range of shoe sizes. The inner volume is separated by a pressure breakable seam approximately at the instep which ruptures when pressure is applied. The seam effectively divides the interior volume into two compartments. One compartment contains a liquid and the other contains a chemical substance. When the seam is ruptured, the liquid flows from one compartment into the other compartment. When the chemical substance combines with the liquid, an endothermic reaction occurs which causes cooling of the foot. The bottom component of the cushioned layer has round dimples into which a dry chemical can be placed. Cooling can be accomplished, for example, by combining ammonium nitrate with water. Adding polyacrylamide into the mix will cause a gel to form in the insert.

### CROSS REFERENCE TO RELATED APPLICATIONS

This Present Application is a non-provisional counterpart of and claims the benefit of and priority to U.S. Provisional Application Ser. No. 60/852,071 filed on Oct. 16, 2006 which is incorporated by reference herein in its entirety. It is also related to co-pending U.S. Non-Provisional application Ser. No. 11/779,307 filed on Jul. 18, 2007 by the Present Applicant (hereinafter, the '307 application) which is also incorporated by reference herein in its entirety. The subject matter for both applications is similar. However, no priority is claimed to said non-provisional application.

### BACKGROUND OF THE INVENTION

Insoles and inserts for shoes have long been popular to increase the comfort level of the shoe wearer. They are primarily designed to cushion or provide additional support for the foot for both medical and non-medical reasons. A fairly recent innovation in shoe inserts is the use of liquid or gel-filled cavities within the insert to provide an adaptive, form-fitting cushion and a more even distribution of force onto the sole of the foot.

This typically involves having a pouch of the liquid or gel within the insert which allows the substance inside to move freely about inside the insert, thus conforming to the shape of the foot. Problems arise with this type of insert construction as it is possible for all the liquid or gel to pool in a single location, thereby negating the benefits of the force-distributive properties of the liquid or gel.

Another innovation to come to footwear is that of temperature control within the shoe. Most common is the use of heat production in a shoe insert. Though there are some employing electrical means of heat production, the most common form is the use of exothermic chemical reactions which provide a steady, relatively long-duration source of heat.

The second, less common form of temperature control within the shoe is by means of decreasing the temperature. Like heated inserts, there are examples that use electrically powered means of cooling the shoe, but there is no prior art employing an endothermic chemical reaction to serve this purpose.

The '307 application discloses a shoe insert for both healing and cooling a person's foot. The insert disclosed therein is filled with chemical materials which, when combined, produce either an exothermic or endothermic reaction. Depending upon the chemical materials used to produce an exothermic reaction, the shoe insert used for heating could be made reusable or disposable. The shoe insert used for cooling would probably be disposable. The shoe insert of the '307 application has round (preferably hemispherical) dimples into which solid chemical materials are inserted and a separate pressure pouch containing a liquid. When the pouch bursts, the chemical materials mix together producing either the exothermic or endothermic reaction.

### SUMMARY OF THE INVENTION

The Present Invention overcomes the drawbacks perceived in the prior art, both of the problems arising from uneven distribution of liquids or gels within shoe inserts and the lack of a chemical means of controlling the temperature of the shoe interior. The present invention is an insole comprising a plurality of wells within the sealed interior of the insole which maintains an even dispersal of a chemical solution responsible for cooling the foot. Unlike the invention disclosed in the '307 application, the Present Invention does not have a breakable interior pouch. Instead, the shoe insert has two sections or compartments separated by a pressure breakable seam which when broken, allow the chemical materials that form the endothermic reaction to mix. The interior composition turns into a cool ice-like gel within minutes of use. When foot pressure is applied, the gel gathers to support the foot arch and to massage the foot. The Present Invention can be used like an ice pack to relieve foot soreness and inflammation. As presently contemplated, the shoe insert remains cold for approximately twenty minutes. Afterwards, it can be refrigerated and reused. It is anticipated that the gel product will be reusable for up to five years.

### BRIEF DESCRIPTION OF THE DRAWINGS

FIG. 1 is a top plan view of the left shoe insert.

FIG. 2 is a top plan view of both shoe inserts.

FIG. 2(a) shows the left insert.

FIG. 2(b) shows the right insert.

FIG. 3 is a side elevation view of the bottom component of the cushioned layer.

### DETAILED DESCRIPTION OF THE INVENTION

This invention, in its preferred embodiment, comprises two layers of a thin, durable material such as vinyl heat sealed along the perimeter of the material roughly in a shape matching the outline of the average foot, though those skilled in the art would also perceive of the benefit of producing a roughly oval shape. The insert comprises two compartments (front-towards toe, and rear-towards heel) separated by a pressure breakable seam. In its currently contemplated mode, the seam has a general V-shape which is positioned so that pressure will burst the seam, thereby allowing the chemical materials to mix. The lower layer is of greater thickness than that of the top layer, such that indentions of approximately 5 mm diameter and 2 mm deep may be placed in the lower level without weakening the integrity of the seam. Between the upper and lower levels in the front compartment reside spherical granules of ammonium nitrate and granules of polyacrylamide evenly distributed throughout most of the front compartment. Similarly the rear compartment is filled with an appropriate volume of water so as to completely saturate the ammonium nitrate and polyacrylamide granules once the seam is broken.

Those skilled in the art may be able to perceive of additional embodiments such as different endothermic chemical reactions or altering the size of the indentations within the bottom layer of the pouch and adjusting the size of the granules to fit accordingly into said indentations.

The user of the preferred embodiment of present invention would burst the seam manually, and with movement gently agitate the mixture inside until the chemicals have been fully inundated with the water. Within the shoe, the application of force from the foot does not displace all of the fluid from the areas receiving the most force due to the indentations in the bottom layer of the insert. The cooling liquid is pooled within the indentations, is thus kept from flowing away from the foot to the sides. This allows for even distribution of the cooling liquid, thereby maximizing the cooling effect of the insert.

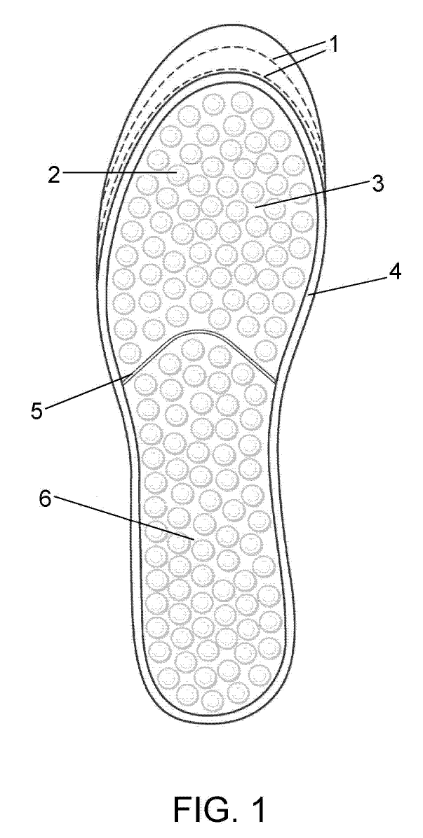

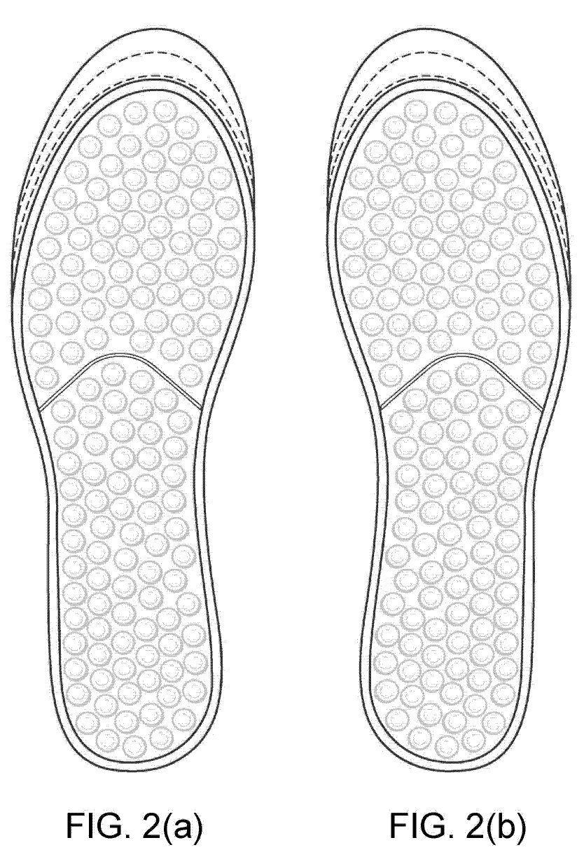

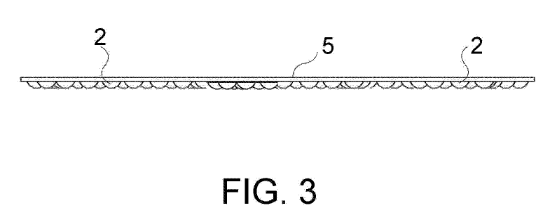

FIG. 1 is a top plan view of the entire shoe insert assembly for the left foot. The right foot shoe insert is identical to the left foot shoe insert shown in FIG. 1, but is the left-right reversed mirror image. Both left and right shoe inserts are shown in FIG. 2. The toe end of the insert on the top layer 1 extends beyond the cushioned region. Alternatively, the extension of the toe end may be on both top and bottom layers. The bottom layer (shown in FIG. 3) comprises round concave pockets 2. The front compartment 3 will hold one tablespoon of ammonium nitrate granules and ¾ teaspoon of small polyacrylamide. A weld 4 (approximately 3 mm) along the perimeter of the insert binding hold the top and bottom layers together. A spot weld 5 (between ½-1 mm), shaped as an inverted “V,” separates the two compartments. The rear area 6 contains water.

At the toe end are break-away strips 1 to adjust the insert to the shoe size. Therefore, the insert may be manufactured either in a single size or in a few sizes such as small, medium, and large. By breaking away the strips 1, the wearer is able to place the insert into a shoe of any size. Clearly, the break-away strips adjust the size of both the top layer as well as bottom layer. In the preferred embodiment, top layer and bottom layer are equal in size and dimensions. They are attached at their perimeters, preferably by a welded seam 4. Breaking off the break-away strips 1 to adjust the size of the insert does not expose the cushioned region since the perimeter attachment of the top and bottom layers is placed where the insert size is smallest. Beyond that position and extending to the end of the toe, the top and bottom layers adhere to each other. The break-away lines are formed so as ease the process of breaking or tearing excess material away from the toe end of the insert. The process of breaking away the strips does not damage the rest of the insert.

The seam 5 ruptures due to the application of pressure to the insert so as to cause liquid to flow from the rear compartment into the front compartment.

The cushioned region is sandwiched between the top layer and the bottom layer. The cushioned region itself comprises three parts:

a top component;

a bottom component; and,

a seam.

The three parts are attached inseparably, preferably with a welded seam 4. In its preferred embodiment, the material used in fabrication of the entire shoe insert is clear (transparent) PVC Vinyl with the upper layer being ˜0.3 mm thick and the bottom layer being ˜0.4 mm thick. Therefore, the top plan view of the entire insert (FIGS. 1 and 2) provides visibility of the entire structure. As previously discussed, the rear compartment contains a liquid (preferably water) that, when the seam ruptures, flows into the front compartment between the top component and the bottom component. The top component is uniform. However, the bottom component (shown in FIG. 3) comprises round (preferably circular) dimples 2 that are depressions in the material. The dimples may be formed by heat pressing into the bottom component. The region between the sandwich may contain a liquid other than that in the pouch or may be dry (not contain any liquid). In the case of a shoe insert designed to cool the foot, measured amounts of chemicals are placed into each of the dimples and water (possibly mixed with a viscous liquid) is placed into the pouch. When the seam 5 ruptures, the water flows into the sandwich and makes contact with the chemicals. The resulting chemical reaction is endothermic, and the shoe insert becomes cold.

Persons having ordinary skill in the art will be able to see that variations in the geometry are obvious and that different chemicals and materials may be inserted into the shoe insert such that when combined, produce an endothermic reaction for cooling the foot.

I claim:

1. A shoe insert shaped like a shoe insole and having a heel end, a toe end, and an instep, said insert having a cushioned layer comprising:
a) a flexible and supple upper component;
b) a lower component; and
wherein:
the upper and lower components are attached and sealed inseparably to each other at their respective perimeters, said attachment forming a volume;
the lower component comprises a plurality of indentations;
the breakable seam is positioned at or near the instep and shares a sealed common boundary with the upper and lower components;
the breakable seam separates the volume into two compartments—a front compartment comprising the toe end, and a rear compartment comprising the heel end;
one of the two compartments contains a liquid;
the other of the two compartments contains a chemical substance;
the common boundary between the pouch and the upper and lower components separates the liquid contents from the chemical substance; and,
the common boundary between the two compartments and the upper and lower components is weaker than the rest of the seal between the upper and lower components, such that when sufficient pressure is applied to the pouch, the common boundary ruptures, thereby permitting the liquid from one compartment to enter into the other compartment and to react with the chemical substance therein.

2. The shoe insert of claim 1 wherein the lower component is flexible and supple.

3. The shoe insert of claim 1 wherein the reaction between the liquid and the chemical substance is endothermic.

4. The shoe insert of claim 3 wherein the chemical substance comprises ammonium nitrate.

5. The shoe insert of claim 3 wherein the chemical substance further comprises polyacrylamide.

6. The shoe insert of claim 4 wherein the chemical substance is inserted into the plurality of indentations.

7. The shoe insert of claim 4 wherein the liquid comprises water.

8. The shoe insert of claim 7 wherein the liquid further comprises a viscous liquid.

9. The shoe insert of claim 1 wherein the combination of the chemical substance and the liquid produces a gel.

10. The shoe insert of claim 1 wherein the cushioned layer is sandwiched between the top and bottom components.

11. The shoe insert of claim 1 further comprising a plurality of break-away strips at the toe end, said strips being joined together such that individual strips can be removed from the insert, in order that the size of the insert can be adjusted to fit a range of shoe sizes by removing one or more of the break-away strips from the insert.
